(19) United States
(12) Patent Application Publication
(10) Pub. No.: US 2005/0202097 A1
(43) Pub. Date: Sep. 15, 2005

(54) Lubricant for the ocular surface

(75) Inventor: Steven Maskin, Tampa, FL (US)

Correspondence Address: PATREA L. PABST;PABST PATENT GROUP LLP, 400 COLONY SQUARE, SUITE 1200, ATLANTA, GA, 30361, US

(73) Assignee: MELBJ Holdings, LLC, Florida

(21) Appl. No.: 11/069,239

(22) Filed: Mar. 1, 2005

Related U.S. Application Data
(60) Provisional application No. 60/552,577, filed on Mar. 12, 2004.
(60) Provisional application No. 60/562,683, filed on Apr. 15, 2004.

(51) Int. Cl. A61K 35/12; A61K 35/78

(52) U.S. Cl. 424/524; 424/757; 424/736

(57) ABSTRACT

A formulation has been developed for treatment of the symptoms of dry eye which incorporates the natural product jojoba wax, or components thereof, to enhance the spreading of the artificial tear and eyedrop as well as stabilize the eyedrop. The improved performance of the jojoba wax supplemented tear relieves irritation and discomfort as well as sharpens the blurred vision.

### CROSS REFERENCE TO RELATED APPLICATIONS

Priority is claimed to U.S. Provisional Application Ser. No. 60/552,577 filed Mar. 12, 2004 and U.S. Provisional Application Ser. No. 60/562,683 filed Apr. 15, 2004.

### BACKGROUND OF THE INVENTION

This invention is generally in the field of ocular lubricants, and in particular relates to a formulation for treatment of the symptoms of dry eye.

The surface of the eye requires constant lubrication for proper function. This includes quality of vision as well as comfort. The eye becomes irritated and vision blurs when inadequately lubricated. This condition is frequently referred to as dry eye. Inadequately treated severe dry eye can lead to cornea scarring, blindness and even loss of the eye. Dry eye is a common condition and many over-the-counter and even prescription therapies are available to mitigate this at times difficult and annoying condition. Many patients are unable to find relief with present therapies.

It is well recognized that the meibomian gland secretions of the eyelid provide the lipid layer of the tear film. The major component of the meibomian gland lipid secretions are wax esters (Driver and Lemp, Meibomian Gland Dysfunction, Surv Ophthalmol 40:343-367, 1996). It is also known that the natural product jojoba is comprised of over 97% wax esters of the long chain variety similar to that of the lipid tear film.

It is therefore an object of the present invention to provide a formulation for alleviating the symptoms of dry eye.

It is a further object of the present invention to provide an over the counter formulation for alleviating the symptoms of dry eye.

### SUMMARY OF THE INVENTION

A formulation has been developed for treatment of the symptoms of dry eye which incorporates the natural product jojoba wax, or components thereof, to enhance the spreading of the artificial tear as well as stabilize the tear film. The jojoba wax tear relieves irritation and discomfort as well as sharpens the blurred vision.

### DETAILED DESCRIPTION OF THE INVENTION

A jojoba liquid wax formulation providing comfort and clarity of vision to patients with dry eye has been developed. The wax esters of the jojoba improve and enhance the spreading, stability and lubricating effect of the artificial tear on the tear film.

I. Formulation

A. Wax

In the preferred embodiment, the formulation contains jojoba wax in an emulsion. The jojoba wax performs as lubricant and evaporation retardant for the tear film. Jojoba wax is a liquid wax composed of long chain wax esters.

The components of the jojoba wax esters include long chain alcohols esterified with long chain fatty acids with a total of 38 to 44 carbon atoms. Exemplary long chain fatty acids include gadoleic, palmitic, palmitoleic, stearic, oleic, linoleic, arachidic, linolenic, eicosenoic, behenic, erucic, lignoceric, lactic, decate, acetic and myristic fatty acids. The fatty acids typically have carbon chains of C12 to C30, with or without various degrees of saturation or unsaturation. The alcohol components of the wax ester contain carbon chains between C16 and C32 with or without various degrees of saturation or unsaturation. The alcohol component may be eicos-11-enol, docos-13-enol, tetracos-15-enol, myristyl alcohol, octyldodecyl stearoyl alcohol or cetyl alcohol.

Jojoba's melting point is about 6° C. It is extracted from seeds and leaves of the jojoba tree (Simmondsia chinensis) cultivated in the desert conditions of Arizona and California as well as Northern Mexico and other locations. The chemical structure does not vary with plant type, growing location, soil type, rainfall or altitude. The oil produced by jojoba lacks triglycerides. It does not contain glycerol combined with fatty acids. Rather the jojoba combines fatty alcohols with fatty acids to produce a vegetable oil which is actually a liquid wax, having its own type of molecular size and shape with unusual anti-evaporative properties which protect the shrub from its severe arid natural habitat. Jojoba wax or the wax esters therein keep the shrub well lubricated and moisturized yet it is non occlusive. The non-occlusive property is related to its porosity. In the shrubs and trees it is derived from, the porosity allows for evaporative exchange of vapors thus cooling the jojoba tree in its hot native climate.

The natural jojoba is 97% wax esters with few impurities. There are no resins, tars, or alkaloids and only a trace amount of saturated wax, alcohols, fatty acids, and hydrocarbons. Jojoba wax is non toxic and biodegradable and is pasteurized to kill microorganisms (National Research Council. 1985. Jojoba: New Crop for Arid Lands, New Material for Industry. National Academy Press, Washington, D.C.). The liquid wax commercially available does not include those solid components of the seed which have toxic effects; the glycosides simmondsin and simmondsin-2-ferulate.

The wax esters are comprised of alcohols esterified with long chain fatty acids with a total of 38 to 44 carbon atoms. The fatty alcohols are predominantly 20 and 22 carbon atoms with one double bond. Its fatty acids are mostly 20:1 (70%), with some 22:1 (20%) and the remainder 18:1 (10%). All double bonds have a cis configuration and are spaced widely apart equidistant from the ester linkage creating an especially stable molecule resistant to oxidation. The cis double bond configuration is also felt to give the jojoba its porosity.

Oils having similar properties to jojoba wax, or its components, may be substituted for the jojoba oil. Jojoba has been identified as chemically similar to sperm whale oil, an unsaturated wax. Sperm whales were sought for their oil throughout the 20^{th} century since it is considered a fine lubricant oil. Due to the near extinction of the sperm whale, alternative lubricants were sought. Although jojoba was known to similar to sperm whale oil since the 1930's, the advanced study of its chemistry was not available until the 1970's and 1980's due to advances in technology. Both are fine lubricants as they are stable at high temperatures and high pressures. However, jojoba is now felt to be a superior lubricant to sperm whale oil (National Academy of Sciences. 1975. Products from Jojoba: A Promising New Crop for Arid Lands. National Research Council Washington D.C.). Another similar oil to sperm whale oil is from the fish Orange Roughy. This oil and other fish oils may be used in place of or in combination with the jojoba.

Jojoba wax is approved by the Food and Drug Administration (“FDA”) for use in cosmetics and other formulations for application around the eyes, although not for direct application to the eye. Jojoba wax is used extensively in the cosmetic industry in up to at least a 10% in water emulsion, in eye makeup remover, as well as for skin and hair products. It is also used in therapeutic massage. Primary eye irritation studies have been performed in rabbits using undiluted refined jojoba liquid wax. Slight irritation was noted which resolved within 24 hours. A 20% natural jojoba wax dropped in rabbit eyes was concluded a nonirritant (Final Report on the Safety Assessment of Jojoba Oil and Jojoba Wax, J Amer College Toxicology, 11 (1), 1992, 57-74.) The Environmental Protection Agency (EPA) in the Federal Register 40 CFR Part 180, 1995 acknowledged the wide distribution of Jojoba in commerce and availability to the general public throughout the United States without any evidence of significant adverse effects to humans or the environment. The Cosmetic Ingredient Review lists Jojoba as safe to use.

Jojoba wax has also been shown to help break down sebum in plugged up sebaceous pores of the skin. It may prove to also be able to break down and unplug the modified sebaceous (meibomian) glands of the lid when applied as a drop or an ointment or other topical therapy.

Jojoba wax also has intrinsic antimicrobial properties which include activity against envelope viruses, mold, fungus and bacteria. U.S. Pat. Nos. 4,585,656 and 6,559,182 describe the efficacy of treating envelope viruses with jojoba wax esters. In vitro experiments in the literature showed jojoba has an intense inhibiting effect on Mycobacterium tubercle bacilli. It may be useful as a prophylactic as well as therapeutic agent to prevent and treat ocular or periocular infections. It may be used as therapy for infection of any part of the eye or adnexal structure.

Other jojoba derivatives which may be incorporated into an ophthalmic delivery system include jojoba esters, jojoba alcohols, and the hydrogenated jojoba solid wax. Jojoba esters are the result of an inter-esterification of various ratios of jojoba liquid wax and hydrogenated jojoba solid wax. The physical consistency ranges from liquid to semi-solid paste or creams. Jojoba solid wax is derived from the hydrogenation and complete reduction of the unsaturated wax esters. It is a hard crystalline wax comparable to beeswax with a melting point of 69° C. and can be prepared in a wax in water emulsion. This wax-in-water emulsion emulsifies easily and may also be used in an ophthalmic preparation. Possible emulsifying agents for the ophthalmic preparation include stearic acid (4%) and triethanolamine (2%). Jojoba alcohols are generated from a sodium reduction of jojoba liquid wax and hydrogenated jojoba solid wax with subsequent additional refinement. Jojobutter-51 is an isomorphous mixture of jojoba liquid wax, partially isomerized jojoba liquid wax and hydrogenated jojoba solid wax (J Amer College Toxicology, 11 (1), 1992). Sulfurization of jojoba results in enhanced lubricant properties which is further enhanced with phosphorus, bromine or chlorine. (Wisniak J The Chemistry and Technology of Jojoba Oil, Am Oil Chemist Society, 1987) and may optimize the lubrication of an ophthalmic tear supplement.

B. Artificial Tears

The wax is mixed with an aqueous solution for application to the eye. Typically the aqueous solution may be sterile water or hypotonic or isotonic saline and will contain buffer to physiological pH, in the range of about 7-7.5. It may also be cell culture media such as Dulbecco's Media (DMEM). It will also contain a surfactant/lubricant/demulcent such as polysorbate 80. Ancillary ingredients to establish the desired tonicity with tears may include electrolytes. Preservatives such as sodium bisulfite, ascorbic acid, alpha-tocopherol, benzalkonium chloride, ethylenediaminetetraacetic acid (EDTA) and chlorhexidine can be used as well as chlorbutanol, sodium perborate and stabilized oxy-chloro complex. Other preservatives include polyquad, polyhexamethyl biguanide, chlorhexidine, propylparabens and methylparabens and others. Other additives may include humectants such as propylene glycol and sorbitol. Representative pH buffers include sodium borate or mono and di-sodium phosphate or other phosphate, carbonate or acetate salts.

The jojoba wax concentration in an aqueous carrier will typically be between 0.001% to 50%. The jojoba in aqueous emulsion may include a second emollient such as mineral or light mineral oil. Other emollients may be used in the emulsion such as white petrolatum, white ointment, paraffin, and beeswax or other wax. These emollients may be used to increase the viscosity of the emulsion. The ratio of jojoba to the second emollient is from greater than 1:5 to 500:1. Jojoba is also available as a clear, water colored refined liquid wax which may also be used as a second emollient in the above ratios.

The formulation may further include a sterol, hydroxycarotenoid or Vitamin A optionally esterified with fatty acids of various chain lengths between C10 and C30. The formulation may also include polar lipids including glycolipids, sphingolipids and/or phospholipids including phosphatidylinositol, phosphatidylethanolamine, sphingomyelin, phosphatidylglycerol, and diphosphatidylglycerol, Triglycerides may also be included.

Suitable lubricants used with the wax ester in a concentration between 0.01% to 20% include cellulose derivatives. Examples of cellulose derivatives include carboxymethylcellulose sodium 0.2 to 2.5%, hydroxyethyl cellulose 0.2% to 2.5%, hydroxypropyl methylcellulose 0.2% to 2.5%, and methylcellulose 0.2% to 2.5%. Other examples of lubricants include Dextran 70, (0.1%), gelatin, 0.01%, glycerin, 0.2 to 1%, polyethylene glycol 300, 0.2 to 1%, polyethylene glycol 400, 0.2 to 1%, polysorbate 80, 0.2 to 5%, propylene glycol, 0.2 to 5%, polyvinyl alcohol 0.1 to 5%, and povidone 0.1 to 5%. These lubricants can increase viscosity of the artificial tear as a mucomimetic and may be added to the formulation. The formulation can be thought of as a tear replacement therapy. Additional mucomimetics include carbomer and hyaluronic acid.

Ophthalmic astringents may also be included. One example is zinc sulfate, 0.25%. A hypertonicity agent may be used such as sodium chloride 2 to 5%. An ophthalmic vasoconstrictor may be used including ephedrine hydrochloride, 0.123%, naphazoline hydrochloride, 0.01 to 0.03%, phenylephrine hydrochloride, 0.08 to 0.2% and tetrahydrozoline hydrochloride, 0.01 to 0.05%.

The eye drop can also include a further emulsifier.

Proteins normally found in the tear may be included in the formulation to further increase stability. These may include amongst others, prealbumin, albumin, lyzozyme, lactoferrin, beta lactoglobulin, IgA as well as lipocalins.

Suitable electrolytes include sodium chloride, potassium chloride, sodium phosphate, potassium phosphate, sodium and potassium sulfates and sodium and potassium bicarbonates. Suitable non electrolytes such as glycerin and sugars such as urea, sorbitol, glucose and sucrose can also be added.

In another embodiment, the jojoba wax, up to 70%, is formulated as an ointment emollient. A suitable carrier includes a mixture of mineral oil and petrolatum in a ratio of about 70% to 30%, paraffin up to 5%, white ointment up to 100%, white petrolatum up to 100%, petrolatum up to 100%, white wax up to 5%, yellow wax up to 5%, colorless jojoba wax up to 50%, lanolin 1 to 10% and anhydrous lanolin 1 to 10%.

The formulation can also be used as a platform to deliver other active agents. Other active ingredients that could be used include anti-glaucoma therapies, antibiotics, antimicrobial peptides, antivirals, antiparasitics, antifungals, antiinflammatories, antihistamines, anti-allergy therapies, hormones such as androgens and others, vitamins, growth factors, cytokines, mucins, surface stimulating drugs, immunomodulators, immune response modifiers, cytokine modifying agents, immunosuppressive agents, antineoplastic agents, eyelash growth stimulators and other medicaments.

Additional classes of additives include lubricants, preservatives, stabilizers, wetting agents, emulsifiers, buffers, and different salts to alter osmotic pressure, as well as solubilizing agents, dispersants, and detergents.

The wax can also be added to artificial tears obtained over the counter (“OTC”). Examples include VISINE™ marketed by Pfizer, REFRESH TEARS™ product line marketed by Allergan, SYSTANE™ marketed by Alcon, GENTEAL™ marketed by Novartis, and OCUCOAT™ marketed by Bausch and Lomb.

II. Methods of Use

In the preferred embodiment, the formulation is administered once to four times a day directly to the eyes of the individual in need thereof. The frequency will vary depending on the severity of symptoms. The formulation may be applied as a drop in the form of an emulsion or suspension, liposome, lotion, ointment, cream, gel, salve or powder and sustained or slow release, as well as eyelid lotion. It may also be used as an eye wash or rinse to irrigate the eye. The formulation may also be applied in a sprayable form. This lubricant will be extremely helpful in eradicating the symptoms of dry eye in the various settings it occurs. This includes the most common settings of age related so called dry eye syndrome, computer related dry eye, dry eye after Lasik, and dry eye associated with reading, driving or watching a movie or television. Patients with contact lens intolerance or who use an ocular prosthesis will also greatly benefit from the enhanced lubrication. Other examples include patients with a history of eye surgery and dry eye. This includes cataract surgery, cornea surgery and cornea transplants. Patients with neurologic disorders such as Bell's Palsy or other neuroparalytic as well as neurotrophic disease will also benefit. Lagophthalmous characterized by an exposed ocular surface which can occur while sleeping or even during waking hours will be improved with the ointment, and/or gel form of this lubricant. Devastating although rare mucous membrane blistering diseases as Stevens Johnson Syndrome are also associated with both a watery and lipid dry eye due to fibrotic changes associated with glandular tissues. The jojoba formulation should be especially helpful to replace lipid and aqueous deficiencies and help relieve suffering to comfort an otherwise extremely painful eye.

Other types of dry eye characterized by plugged, inflamed and/or dysfunctional sebaceous glands of the lid known as meibomian gland dysfunction should also be mproved with use of this formulation applied to the eyelids.

Patients with eye infections of the lid, conjunctiva, cornea and tear apparatus and lacrimal gland should also benefit with application of this formulation in one or more forms to the eyelids, conjunctiva, and cornea as well as tear film and other adnexal structures including lacrimal gland, and tear outflow system including puncta, canaliculi, and lacrimal sac.

In preliminary studies on skin, Jojoba wax has been shown to relieve pain and reduce swelling from superficial thermal and chemical burns. There may also be a therapeutic effect on ocular burns.

The formulation can also be used to prevent, treat or alleviate the symptoms of envelope viruses including herpes simplex keratitis, and varicella zoster keratitis which causes chicken pox and shingles. Other viral infections of the eye that may be treated include human herpes virus 8 (HSV 8), Kaposi sarcoma as well as Epstein-Barr virus, cytomegalic inclusion virus (CMV) and Human Immunodeficiency Virus (HIV).

Non-ocular uses of the formulation include use to treat or prevent accumulation of ear canal wax, treatment of vaginal dryness or other symptoms of perimenopausal dryness, moisturizing dry nasal mucosa or where the patient has a sinus condition, including inflammation or infection.

### EXAMPLES

In a preferred embodiment, the formulation contains 0.5-5% jojoba wax, most preferably 0.5 to 2% jojoba, 1% polysorbate 80 in a aqueous buffered saline based liquid wax emulsion.

The 2% jojoba formulation was administered to a total of 16 volunteer individuals with different types of irritated eyes. The drop was reported to be extremely comfortable for all individuals without causing visual blur.

Three volunteers had painful dry eye after Lasik. None of the conventional therapies had helped them thus far. For PC, AS, and KA, relief was immediate and lasted about 8-10 hours.

For TB who said his irritation was allergic in nature, none of the presently available OTC drops had helped relieve his severe symptoms. One drop of the jojoba wax formulation applied to each eye relieved all symptoms for the entire day.

For JR who said his eyes are always irritated in the morning, get red and stay red for hours and who has yet to find a comfortable and effective OTC eyedrop, one drop of the jojoba wax formulation applied to each eye eliminated the red eyes and comforted his eyes for the entire day.

Two individuals (RD and AM) used the jojoba wax formulation in the setting of soft contact lens wear and found its comforting properties to be truly unique. They enjoyed instant relief of eye discomfort which lasted the entire day.

One individual (ST) used the jojoba wax formulation in the setting of rigid contact lens wear and also had instant relief of eye irritation lasting the whole day.

In summary, the volunteers were extremely pleased by the comfort, immediate and lasting relief of the jojoba wax formulation.

Three additional patients (HK, LF, and IM) with cornea erosions were placed on this formulation using 1% jojoba wax. The drop was used four times per day. The drop was well tolerated, and was found to be soothing and very comfortable. Within one to two weeks the erosions were markedly and almost completely resolved.

A formulation consisting of 5% jojoba in aqueous with additional 0.05% white petrolatum USP was created using a heating stir plate and was placed in the right eye of 6 volunteers. For MB, MH, DN, HL, AM, and SM the drop was well tolerated, comfortable and felt thicker than 5% jojoba in aqueous emulsion without the petrolatum.

The formulation was also evaluated on two volunteers using lipid tear interferometry. A drop of the formulation was placed in one eye and an artificial aqueous tear in the other. The interferometry pattern showed thick blue waves of liquid wax quickly mixing with the volunteer's own lipid tear within seconds. The resultant lipid tear pattern showed a healthy enhanced film at least three hours later. Breakup times were also prolonged therapeutically in the eye receiving the emulsion compared to the fellow eye.

Modifications and variations of the present invention will be obvious to those skilled in the art from the foregoing detailed description and are intended to come within the scope of the following claims. All references herein are expressly incorporated by reference.

1. An ophthalmic composition comprising a effective amount of a wax selected from the group consisting of jojoba wax or a component or derivative thereof, sperm oil or orange roughy oil, to lubricate the eye.

2. The ophthalmic composition of claim 1 comprising an emulsion of jojoba wax or derivative thereof, or a component thereof, with a material selected from the group consisting of an ophthalmic lubricant, a surfactant, an emulsifier, a viscosity enhancer, and combinations thereof, in a water based emulsion, wherein the wax or component thereof is in an amount effective to increase lubrication or tear stability and reduce evaporation of the tear when applied to the surface of the eye.

3. The ophthalmic composition of claim 1 wherein the component is a wax ester and/or alcohol esterified with long chain fatty acids with a total of 12 to 62 carbon atoms.

4. The ophthalmic composition of claim 1 comprising an aqueous carrier, in which the concentration of jojoba oil is between 0.001% to 50%.

5. The ophthalmic composition of claim 3 wherein the long chain fatty acids are selected from the group consisting of gadoleic, palmitic, palmitoleic, stearic, oleic, linoleic, arachidic, linolenic, eicosenoic, behenic, lignoceric, lactic, decate, acetic and myristic fatty acids.

6. The ophthalmic composition of claim 3 wherein the fatty acids have carbon chains of C12 to C30, with or without various degrees of saturation or unsaturation.

7. The ophthalmic composition of claim 3 wherein the alcohol component of the wax ester contains carbon chains between C16 and C32 with or without various degrees of saturation or unsaturation.

8. The ophthalmic composition of claim 7 wherein the alcohol component is selected from the group consisting of eicos-11-enol, docos-13-enol, tetracos-15-enol, myristyl alcohol, octyldodecyl stearoyl alcohol and cetyl alcohol.

9. The ophthalmic composition of claim 3 further comprising a sterol, hydroxycarotenoid or Vitamin A, optionally esterified with fatty acids of chain lengths between C10 and C30.

10. The ophthalmic composition of claim 3, comprising wax ester in a concentration between 0.001% to 50%.

11. The ophthalmic composition of claim 2, wherein the lubricant or viscosity enhancer is a cellulose derivative.

12. The ophthalmic composition of claim 11 wherein the cellulose derivative is selected from the group consisting of carboxymethylcellulose sodium 0.2 to 2.5%, hydroxyethyl cellulose 0.2% to 2.5%, hydroxypropyl methylcellulose 0.2% to 2.5%, and methylcellulose 0.2% to 2.5%.

13. The ophthalmic composition of claim 2, wherein the lubricant or viscosity enhancer is selected from the group consisting of Dextran 70, (0.1%), gelatin, 0.01%, glycerin, 0.2 to 1%, polyethylene glycol 300, 0.2 to 1%, polyethylene glycol 400, 0.2 to 1%, polysorbate 80, 0.2 to 5%, propylene glycol, 0.2 to 5%, polyvinyl alcohol 0.1 to 5%, povidone 0.1 to 5%, carbomer or hyaluronic acid.

14. The ophthalmic composition of claim 1, combined with an astringent.

15. The ophthalmic composition of claim 14 wherein the astringent is zinc sulfate, 0.25%.

16. The ophthalmic composition of claim 1, combined with an ophthalmic vasoconstrictor selected from the group consisting of ephedrine hydrochloride, 0.123%, naphazoline hydrochloride, 0.01 to 0.03%, phenylephrine. hydrochloride, 0.08 to 0.2% and tetrahydrozoline hydrochloride, 0.01 to 0.05%.

17. The ophthalmic composition of claim 14 combined with a vasoconstrictor.

18. The ophthalmic composition of claim 1 combined with a lubricant, vasoconstrictor and astringent.

19. The ophthalmic composition of claim 2 further comprising an emulsifier.

20. The ophthalmic composition of claim 1 comprising an aqueous carrier and electrolytes selected from the group consisting of sodium chloride, potassium chloride, sodium phosphate, potassium phosphate, sodium and potassium sulfates and sodium and potassium bicarbonates or non electrolyte selected from the group consisting of glycerin, urea, sorbitol, glucose and sucrose.

21. The ophthalmic composition of claim 1 further comprising a semi-solid ointment or cream or an emulsion with a second emollient selected from the group consisting of a mixture of mineral oil and petrolatum in a ratio of about 70% to 30%, paraffin up to 5%, white ointment up to 100%, white petrolatum up to 100%, petrolatum up to 100%, white wax up to 5%, yellow wax up to 5%, mineral oil up to 50%/, light mineral oil up to 50%, lanolin 1 to 10% and anhydrous lanolin 1 to 10%, colorless jojoba wax up to 50%, and combinations thereof, wherein the ratio of the second emollient to jojoba wax must be less than 5:1.

22. The ophthalmic composition of claim 2 comprising a polar lipid or oil selected from the group consisting of glycolipid, sphingolipid, phospholipid, and triglyceride.

23. The ophthalmic composition of claim 1 further comprising agents selected from the group consisting of antivirals, antibiotics, antifungals, antiparasitic agents, hormones, growth factors, cytokines, mucins, surface stimulating drugs, vitamins, immunomodulators, immunosuppressive agents, immune response modifiers, cytokine modifying agents, anti-inflammatory, anti-allergy and anti-glaucoma, antineoplastic agents, and eyelash growth stimulators.

24. The ophthalmic composition of claim 1 further comprising agents selected from the group consisting of lubricants, preservatives, stabilizers, wetting agents, emulsifiers, buffers, salts to alter oncotic pressure, solubilizing agents, dispersants, and detergents.

25. The ophthalmic composition of claim 1 further comprising proteins to improve tear stability selected from the group consisting of prealbumin, albumin, lyzozyme, lipocalins, beta lactoglobulin, lactoferrin and IgA.

26. A method of use of a composition comprising a effective amount of a wax selected from the group consisting of jojoba wax or a component or derivative thereof, sperm oil or orange roughy oil, to lubricate a tissue surface in need thereof.

27. The method of claim 26 wherein the patient has dry eye due to allergies.

28. The method of claim 26 wherein the patient has had eye surgery.

29. The method of claim 26 wherein the patient wears contact lenses.

30. The method of claim 26 wherein the patient has a bacterial or fungal infection.

31. The method of claim 26 for application to the nasal mucosa.

32. The method of claim 26 for application to the lid margin to unblock the modified sebaceous glands of the eyelid known as meibomian glands.

33. The method of claim 26 to lubricate the eye, as a contact lens lubricant for either soft or rigid lenses.

34. The method of claim 26 for application and use with ocular prosthesis.

35. The method of claim 26 for application to the outer ear and ear canal to treat or prevent ear wax accumulation.

36. The method of claim 26 for treatment or prevention of vaginal dryness or other symptoms of perimenopausal dryness.

37. The method of claim 26 to provide relief wherein the patient has dry nasal mucosa.

38. The method of claim 26 to provide relief wherein the patient has a sinus condition.

39. The method of claim 26 to provide relief wherein the patient has meibomian gland dysfunction.

40. The method of claim 26 wherein the formulation is selected from the group consisting of solutions, suspensions, liposomes, lotions, creams, ointments, emulsions, sprays, salves, powders, and eye rinse for irrigation of the eye.

41. The method of claim 26 to treat or prevent recurrences as well as the initial viral infection.

42. The method of use of claim 26 to treat or prevent allergies, microbial infections from bacteria, molds, fungi, parasites and viruses in the eye when there is no dry eye present.

43. The method of claim 26 to rewet or increase comfort in the setting of contact lens use or after eye surgery in the absence of dry eye.
